(19) United States
(12) Patent Application Publication
(10) Pub. No.: US 2023/0310852 A1
(43) Pub. Date: Oct. 5, 2023

(54) TREATMENT OF ISCHEMIA

(71) Applicant: Sky Medical Technology Ltd, Buckinghamshire (GB)

(72) Inventors: Bernard Ross, Buckinghamshire (GB); Duncan Bain, Kings Langley, Hertfordshire (GB); Arthur Tucker, Leytonstone, London (GB)

(73) Assignee: Sky Medical Technology Ltd., Buckinghamshire (GB)

(21) Appl. No.: 18/020,564

(22) PCT Filed: Aug. 18, 2021
(86) PCT No.: PCT/EP2021/072941

§ 371 Date: Feb. 9, 2023

(30) Foreign Application Priority Data
Aug. 18, 2020 (GB) 2012880.7

(51) Int. Cl. A61N 1/36

(52) U.S. Cl. CPC A61N 1/36025; A61N 1/36003; A61N 1/36031; A61N 1/0456

(57) ABSTRACT

Methods for improving patient recovery after cerebral ischaemia are described. The methods include administering an electrical stimulus to a nerve of the patient innervating opposed leg muscles sufficient to cause isometric contraction of said muscles. The isometric contraction may result in improved or increased cerebral blood flow, and in some embodiments said electrical stimulation may be sufficient to alter electrical activity in the brain.

### FIELD OF THE INVENTION

The present invention relates to a method of treatment of ischemia; and more particularly to methods and techniques for improving patient outcomes in cerebral ischemia after stroke.

### BACKGROUND TO THE INVENTION

One factor which potentially influences patient outcome in cerebral ischemia after stroke is the regulation of blood flow in the brain. Stroke patients may have reduced ability to regulate blood flow to maintain cerebral blood perfusion, which in turn results in increased tissue damage and potentially further ischemia. Therapies aimed at restoring cerebrovascular regulation after ischemia offer the opportunity to improve cerebral perfusion and limit ischemic injury. Improved cerebral perfusion may also reduce the risk of further ischaemic events, and/or reduce the extent of cell death and spread of infarction.

One such potential therapy is sequential external counterpulsation - essentially, inflatable cuffs are placed on a subject’s leg, and inflated and deflated in time with diastole and systole respectively. Studies in healthy patients indicate that such treatments may increase cerebral blood flow. See, for example, “Sequential External Counterpulsation Increases Cerebral and Renal Blood Flow”, Applebaum et al, Am Heart J . 1997 Jun;133(6):611-5. doi: 10.1016/s0002-8703(97)70161-3. Early clinical trials in ischemic patients suggest that there may be some effect on recovery from stroke, although the data is inconclusive - see, for example, “Feasibility and Safety of Using External Counterpulsation to Augment Cerebral Blood Flow in Acute Ischemic Stroke-The Counterpulsation to Upgrade Forward Flow in Stroke (CUFFS) Trial”, Guluma et al, J Stroke Cerebrovasc Dis. 2015 Nov;24(11):2596-604. doi: 10.1016/j.jstrokecerebrovasdis.2015.07.013. Epub 2015 Sep 4, which concludes “ECP was safe and feasible to use in patients with acute ischemic stroke. It was associated with unexpected effects on flow velocity, and contemporaneous improvements in NIHSS score regardless of pressure used, with a possibility that even very low ECP pressures had an effect. Further study is warranted.”

Separately, the use of intermittent pneumatic compression in immobile stroke patients has been investigated for reducing the risk of deep vein thrombosis (DVT). The CLOTS 3 trial (“CLOTS (Clots in Legs Or sTockings after Stroke) Trials Collaboration. Effectiveness of intermittent pneumatic compression in reduction of risk of deep vein thrombosis in patients who have had a stroke (CLOTS 3): a multicentre randomised controlled trial” Lancet 2013; 382: 516-24) concluded that intermittent pneumatic compression is effective in reducing the risk of DVT in immobile stroke patients. The compression stockings used in this study were not synchronised with heart beat.

A response to the CLOTS 3 trial from Barer (Lancet 2013; 382: 1481) queried whether a reduction in 30-day mortality seen in the trial may have been a result of the simpler intermittent pneumatic compression achieving similar effects to the sequential external counterpulsation system by redistributing the venous blood pool.

One example of a device, intended at least in part as a therapy for prevention or reduction of DVT, is the Geko system produced by Firstkind Ltd. The system is described in detail in international patent applications WO2006/054118 and WO2010/070332. In brief, however, an electrical stimulation device is used to stimulate the common peroneal nerve at a location which has the effect of causing simultaneous contraction of anterior and posterior muscle groups leading to isometric contraction. For example, stimulation may take place at the fibula head or in the popliteal fossa, with the device being located in line with the outer lateral hamstring tendon/outer biceps femoris tendon, either above or below the crease of the knee. This contraction activates the calf and foot muscle pumps leading to increased blood circulation in the limb, potentially reducing the risks of DVT among other beneficial effects from improved blood circulation.

There is a need for improved methods and techniques for assisting recovery of patients with cerebral ischemia following stroke.

Potential further factors which may influence recovery of patients from cerebral ischaemia include sensory stimulation, neuromodulation, or enhancing neuroplasticity after stroke. In certain embodiments, it would be advantageous for certain methods and techniques to also include one or more of these treatment modalities in addition to modulating blood flow.

### SUMMARY OF THE INVENTION

According to a first aspect of the present invention, there is provided a method of improving recovery of a patient from cerebral ischemia, the method comprising administering an electrical stimulus to a nerve of the patient innervating opposed leg muscles sufficient to cause isometric contraction of said muscles.

The present inventors have determined, surprisingly, that electrical stimulation of the nature previously used to improve blood flow in the limb has the further effect of increasing cerebral blood flow. This therefore opens up the possibility of using the electrical stimulation to improve patient recovery from cerebral ischemia.

A further surprising effect of the use of electrical stimulation is that the stimulation may also result in alteration in electrical activity in the brain, even when the nerve being stimulated innervates the leg muscles. The inventors believe that this may also result in improved patient recovery, for example as cerebral electrical activity is regulated and recovered post-ischemia.

By “improved recovery” is meant an improvement in patient outcome compared to a patient with the same or similar initial conditions but who has not received the therapy. For example, the patient may have an improvement in motor or cognitive function compared with a control patient (or compared with the same patient immediately prior to treatment). Standard tests for motor or cognitive function in stroke patients are known, and may be used here to determine whether improved recovery occurs. As another example, reduction in cerebral cell death compared with a control patient may represent improved recovery; as may increased neuroplasticity in recovery of functions post stroke.

In general, an improved recovery may be a consequence of two alternative routes (which may be combined). The first is a reduction in the risk of further ischaemic events; if further events are less likely to happen, then the patient is able to enjoy a longer period of uninterrupted recovery of cerebral function. The second is stimulation of the cerebral neurons to recover function from neuroplasticity and reassignment of neuronal function from inactive neurons. We believe that either or both of these routes can be achieved using the present invention; primarily enhanced blood flow will achieve a reduction in risk of further events, while electrical stimulation of nerves will result in stimulating neuroplasticity. However, we do not rule out that at least some contribution to both routes comes from both treatment modalities, and combining the two into a single treatment may be beneficial.

In some embodiments, the enhanced or improved cerebral blood flow may be identified as or may manifest as an improvement in regulation of cerebral perfusion pressure (CPP). CPP is determined by two factors, the mean arterial pressure (MAP), and intracranial pressure (ICP). In healthy individuals, CPP is controlled and kept at a constant level by cerebral autoregulation, which in simple terms increases or decreases ICP with a corresponding increase or decrease of MAP; hence CPP remains constant within a certain range of fluctuation. In a stroke, cerebral autoregulation is impaired, so a direct increase in the blood flow by increasing MAP could increase the CPP. Hence, the present invention may be beneficial as a means to improve, restore, or assist cerebral autoregulation in a patient.

In certain situations, treatment may be divided into an acute phase, and a post-acute phase. The relative importance of the two recovery routes differs during each phase. In particular, improved blood flow is considered particularly important during the acute phase (that is, immediately post-ischaemic event), so as to reduce the risk of further events, while altered electrical activity is considered more important in the longer post-acute phase, to allow a longer period of neural recovery. In stroke patients, the final infarct size develops over some time - typically up to 18-24 hours. A part of the tissue dies immediately (the infarct core) surrounded by dying tissue called the ischaemic penumbra. Immediate attention in the acute phase (or hyperacute phase) can reduce the development of the ischaemic penumbra, thereby reducing the final infarct size and potentially leading to improved patient outcomes.

Hence, a further aspect of the invention provides a method of improving recovery of a patient from cerebral ischemia, the method comprising administering an electrical stimulus to a nerve of the patient innervating opposed leg muscles sufficient to cause isometric contraction of said muscles, said isometric contraction resulting in improved or increased cerebral blood flow, and said electrical stimulation being sufficient to alter electrical activity in the brain.

In certain embodiments of the invention, the improvement in blood flow and the alteration of electrical stimulation in the brain may be decoupled; that is, one set of stimulation parameters / conditions results in isometric muscle contraction thereby improving cerebral blood flow (and/or improving regulation of CPP); and a second set of stimulation parameters / conditions results in altered electrical activity in the brain. The second set of parameters / conditions may be separated in space (eg, at a different location on the body), in time (eg, at a different treatment session), or both, from the first set of parameters / conditions.

The method may further comprise administering a separate treatment to improve neuroplasticity and/or to alter electrical activity in the brain. This may be administered together with or separately from the treatment of the present invention.

A yet further aspect of the invention provides a method for selecting a patient for therapy to aid recovery from cerebral ischaemia, the method comprising:
a) identifying a patient who has experienced cerebral ischaemia;
b) administering an electrical stimulus to a nerve of the patient innervating opposed leg muscles sufficient to cause isometric contraction of said muscles;
c) monitoring cerebral blood flow prior to and during and/or after said administration of electrical stimulus; and
d) selecting the patient for continued therapy in the event that cerebral blood flow increases during and/or after said administration.

The continued therapy may be further therapy comprising administering an electrical stimulus to a nerve of the patient innervating opposed leg muscles sufficient to cause isometric contraction of said muscles. The continued therapy may be an alternative and/or an additional therapy which results in altered electrical activity in the brain,

A yet further aspect of the invention provides a method for selecting a patient for therapy to aid recovery from cerebral ischaemia, the method comprising:
a) identifying a patient who has experienced cerebral ischaemia;
b) administering an electrical stimulus to a nerve of the patient innervating opposed leg muscles sufficient to cause isometric contraction of said muscles;
c) monitoring brain electrical activity prior to and during and/or after said administration of electrical stimulus; and
d) selecting the patient for continued therapy in the event that brain electrical activity alters during and/or after said administration.

A yet further aspect of the invention provides a method for selecting a patient for therapy to aid recovery from cerebral ischaemia, the method comprising:
a) identifying a patient who has experienced cerebral ischaemia;
b) administering an electrical stimulus to a nerve of the patient innervating opposed leg muscles sufficient to cause isometric contraction of said muscles;
c) monitoring cerebral blood flow and brain electrical activity prior to and during and/or after said administration of electrical stimulus; and
d) selecting the patient for continued therapy in the event that cerebral blood flow increases and brain electrical activity alters during and/or after said administration.

The following remarks apply to all aspects of the invention, unless otherwise noted.

Preferably the method is used as soon as possible after the patient experiences cerebral ischaemia. Ideally, this is within the same day; although it may be within 2, 3, 4, 5 days, or within 1 or more weeks. In certain embodiments the method may be used within 6 months of the patient experiencing cerebral ischaemia, more preferably within 5 months, 4 months, 3 months, 2 months, or 1 month.

The method may further comprise providing additional electrical stimulation to the brain. For example, by transcranial electrical stimulation; and/or by stimulating one or more nerves in the patient sufficient to alter electrical activity in the brain.. Alternatively, or in addition, the method may comprise administering transcranial magnetic stimulation (TMS) to the patient. It has been shown that TMS can help in restoration of motor functions after stroke (see, for example, Hoyer EH, Celnik PA. Understanding and enhancing motor recovery after stroke using transcranial magnetic stimulation. Restor Neurol Neurosci. 2011;29(6):395-409. doi:10.3233/RNN-2011-0611).

In certain aspects, the invention may further comprise administering a therapeutic compound or preparation for treating stroke. For example, therapeutic compounds or preparations may include thrombolytic medicines (for example, alteplase [tPA, rtPA]); antiplatelet medications (for example, aspirin, clopidogrel, dipyridamole); anticoagulant medications (for example warfarin, apixaban, dabigatran, edoxaban, rivaroxaban, heparin); anti-hypertensive medications (for example, thiazide diuretics, angiotensin-converting enzyme (ACE) inhibitors, calcium channel blockers [eg, nimodipine], beta-blockers, alpha-blockers); anti-cholesterol medications (for example, statins).

The leg muscles are preferably the calf muscles, although in certain embodiments of the invention, stimulation of the ankle and/or foot musculature may instead or in addition be used. The leg muscles are preferably involved in a musculovenous pump; for example, the calf, foot, and/or thigh pumps. Improvement in blood circulation from such pumps is not restricted to the leg.

It is believed that the major improvement in blood flow according to the present method is from activation of the musculovenous pump. However, the inventors also believe that a certain amount of blood flow improvement arises as a consequence of electrical stimulation of the skin or near surface subcutaneous circulatory network. Accordingly, it may be possible for the method to further comprise administering electrical stimulation to the skin to thereby improve blood flow (and/or improve cerebral autoregulation of CPP).

Stimulation of the lateral popliteal nerve, in the region of the popliteal fossa, has the advantage of initiating the contraction of both posterior and anterior lower limb muscle groups from a single stimulation point. Such simultaneous stimulation results in isometric contraction; hence the ankle and knee joints would not be typically mobilised. Stimulation of the lateral popliteal also elicits contraction of the foot muscles and hence the so-called “foot-pump”.

The method preferably comprises repeatedly administering an electrical stimulus to the nerve.

A typical electrical stimulus may be at a current of between 0 to 100 mA, preferably 0 to 50 mA, more preferably 1 to 40 mA, and most preferably between 1 to 20 mA. Other examples of stimulus currents include between 15 and 30 mA.

The stimulus may be an AC waveform, although it is preferably a DC waveform, more preferably a pulsed DC waveform. The stimulus may have a frequency of 0.01 to 100 Hz, preferably 0.1 to 80 Hz, more preferably 0.1 to 50 Hz; and more preferably still 0.1 to 5 Hz. The most preferred frequencies are 0.5-5 Hz, 1-5 Hz, preferably 1-3 Hz; for example, 1, 2 or 3 Hz, and most preferably 1 Hz. The precise desired frequency may depend on the stage of patient being treated (for example, acute phase vs post-acute phase).

Specific examples of preferred stimuli include 20 mA, at a frequency of 5 Hz, 30 mA at 3 Hz, and 28 mA at 1 Hz. Other stimuli may of course be used. Most preferred specific stimuli are square wave pulses at a rate of 1 Hz, of a constant current of 27 mA, 38, or 54 mA. Pulse widths may be between 35 us and 560 us.

The stimulus may be applied for a duration between 0 and 1000 ms, between 100 and 900 ms, between 250 and 750 ms, between 350 and 650 ms, or between 450 and 550 ms. In certain embodiments, the stimulus may be applied for up to 5000 ms, up to 4000 ms, up to 3000 ms, or up to 2000 ms. Other durations may be used; again this may depend on the details of the patient or the mode of action intended. Other preferred durations include from 70 to 600 ms. In certain embodiments, yet shorter durations may be used, for example from 25 us to 800 us.

The duration of treatment will depend on the patient response.

Characteristics of the stimulus may vary over time. For example, a single stimulus may increase in current over the duration of the stimulus. Preferably the increase is gradual up to a peak; the stimulus may then either be maintained at the peak; terminate at the peak; or decrease in a gradual manner. Alternatively, where repeated stimuli are applied, characteristics of the stimuli may vary between different stimuli. For example, successive stimuli may be applied at increasing levels of current. Again, these successive stimuli may increase up to a peak gradually, followed by maintenance at that peak, or decrease from the peak. A cycle of increasing stimuli may be repeated a number of times. In preferred embodiments, each stimulus is a single pulse, rather than multiple brief pulses.

In certain embodiments, the stimulus characteristics vary between the acute phase of the treatment and the post-acute phase. In particular, we believe that a low current is most appropriate for the acute phase of treatment, where the prime concern is increasing blood flow by neuromuscular electrical stimulation to activate the musculovenous blood pumps. In the post-acute phase of treatment, a higher current may be appropriate, where the prime concern is altering electrical activity in the brain as a consequence of electrical stimulation of the nerve.

Determining appropriate stimulation parameters may be achieved for example by administering a stimulus with a given set of parameters, and then monitoring for a particular desired response (for example, an increase in cerebral blood flow, and/or an electrical response in the brain). If no or an inadequate response is seen, then the stimulation parameters may be adjusted, and the process repeated. For example, stimulation may begin at a low current which may then be increased until a response is observed.

A yet further aspect of the invention provides a method of treatment of a patient having dementia, the method comprising administering an electrical stimulus to a nerve of the patient innervating opposed leg muscles sufficient to cause isometric contraction of said muscles, said isometric contraction resulting in improved or increased cerebral blood flow, and said electrical stimulation being sufficient to alter electrical activity in the brain. The present inventors have surprisingly determined that - similar to recovery from ischaemia - the combination of increased cerebral blood flow and altered electrical activity in the brain may result in an improvement in symptoms of dementia patients. The patient may have young onset dementia; vascular dementia; mixed dementia; frontotemporal dementia; Lewy body dementia; Creutzfeldt-Jakob disease; Alzheimer’s disease; Huntington’s disease; Niemann-Pick disease type C; Parkinson’s disease; posterior cortical atrophy; progressive supranuclear palsy; or Wernicke-Korsakoff syndrome. Other features of this aspect of the invention - for example, preferred stimulation parameters etc - are in line with those of the other aspects of the invention. This aspect of the method may further comprise administering one or more medications suitable for treatment of dementia. For example, the medication may be a cholinesterase inhibitor (for example, donepezil, rivastigmine, or galantamine); or a glutamate regulator, for example memantine.

Without wishing to be bound by theory, it may be that the combination of effects results in supporting brain perfusion when perfusion is suboptimal; hence the observed outcomes in treatment of both stroke and dementia patients.

The devices described in WO2006/054118 and WO2010/070332 (the contents of which are incorporated by reference) are certain examples of devices which may be used to administer electrical stimulation to a patient. In some embodiments of the invention, the electrical stimulation may be administered by means of a device such as those described in WO2006/054118 and/or WO2010/070332. In preferred embodiments of the invention, the electrical stimulation is administered by means of a device comprising positive and negative electrodes for administering an electrical stimulus to a nerve which innervates opposed leg muscles of a patient; a power supply connectable to the electrode; and a control means for activating the electrodes to administer an electrical stimulus to said nerve sufficient to cause the muscles to contract isometrically.

In some embodiments the device includes one or more positive electrode(s) which is shaped and located either side of a negative electrode. For example, the positive electrode may be generally C-shaped or U-shaped, and the negative electrode may be located within the opening of the C or the U. In preferred embodiments the device includes a single negative electrode and two or more positive electrodes; and more preferably the total area of the positive electrode(s) is greater than that of the negative electrode. It has been found that such an arrangement provides a higher charge density at the motor point, and greater capacitance overall.

The electrodes may be silver electrodes.

The electrodes may be continuous, or may include holes - for example, the electrodes may be solid electrodes, or may be in the form of a mesh.

Preferably the control means is configured to administer an AC electrical stimulus. Preferably the waveform of the current is asymmetric; conveniently the waveform provides an initial (positive) pulse of large magnitude and short duration, followed by a (negative) pulse of small magnitude and long duration. The area under the curve of the two pulses will be equal. In one embodiment, the initial pulse is of a generally square waveform.

In preferred embodiments, the device comprises a flexible substrate on which are mounted the electrodes, the power supply, and the control means. The control means may be, for example, a PCB configured to activate the electrodes as appropriate. The power supply may be an electrical cell. The substrate is preferably flexible, but not stretchable - this reduces the risk of the electrodes cracking or breaking. For example, the substrate may be a thermoplastic elastomer.

The electrodes may be directly printed onto the substrate, by conventional printing means (for example pad or tampo printing). Similarly, conductive tracks may also be printed onto the substrate if desired.

The substrate may be in the form of an elongate strip or tongue, with the electrodes spaced along the strip. Such an arrangement may require a conductive track to be placed from the power supply to the further electrode, passing close to the nearer electrode. In such arrangements, the device may further comprise one or more insulative strips or regions arranged to separate the conductive track from the nearer electrode; insulative strips may also or instead be arranged along the edges of the strip to prevent current leaking outside the area of the strip. Alternatively, or in addition, the substrate may comprise a recessed groove within which a conductive track may be located; thereby serving to separate the track from the electrode.

The device further comprises a conductive gel overlying the electrodes. The gel is preferably in a single piece overlying both electrodes, for ease of manufacture as well as structural integrity. We have determined that a single piece of gel may be used, based on the bulk resistivity of the material and geometry, so that leakage resistance is much greater than delivery resistance. Examples of gels which may be used include hydrogel or silicone.

The device may include a press button for activating or deactivating the device. The control means may be configured to provide a plurality of activation modes (for example, with different stimulation characteristics); the press button may be used to cycle through these modes. The device may include a display means, such as a light or an LED, to indicate the selected activation mode.

### BRIEF DESCRIPTION OF THE FIGURES

FIGS. 1 and 2 show views of an example of a device for administering electrical stimulus to a nerve innervating opposed leg muscles of a patient, such as may be used in the methods of the present invention.

FIG. 3 shows brain stimulation levels in healthy volunteers with the GEKO device.

### DETAILED DESCRIPTION OF THE INVENTION

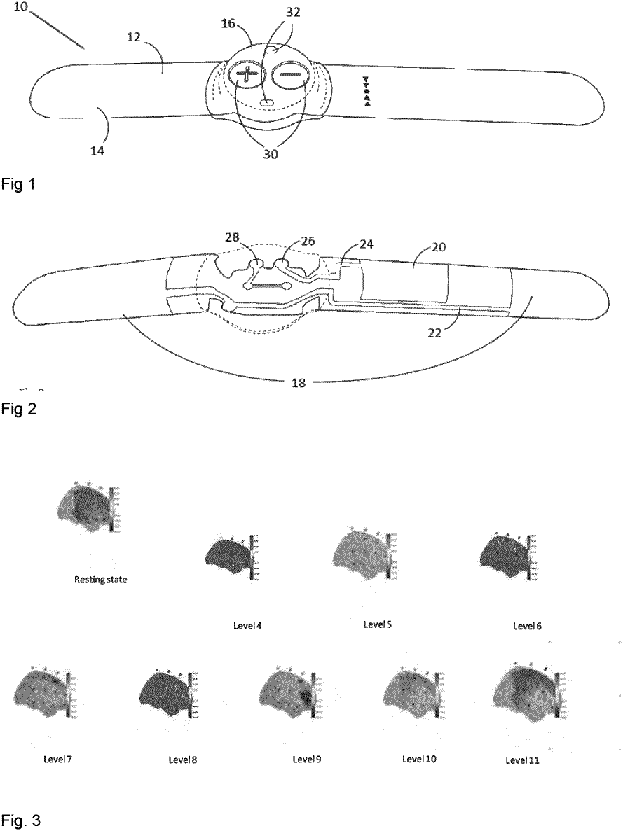

A device 10 which may be used to administer electrical stimulation to a nerve innervating opposed leg muscles of a patient is shown in FIGS. 1 and 2. FIG. 1 shows the outward appearance of the device, while FIG. 2 illustrates the electrodes and electrical pathways formed on the device. The device 10 comprises a flexible, non-stretchable thermoplastic elastomer substrate 12 which includes a moulded recess 16 centrally located between two elongate tongues 14, each of which carries one or more electrodes 18, 20. In some embodiments, the device may include a single elongate tongue 14 at one end, and a moulded recess 16 at the other.

On the tongue 14 are printed positive 18 and negative 20 electrodes. The positive is slightly larger than the negative. Each electrode includes a conductive track 22, 24 leading from the electrode to a respective contact point 26, 28 located in the recess 16.

Within the recess 16 are placed an electrical cell (not shown), and a PCB (not shown) including suitable circuitry to control the electrodes. Together with the conductive tracks 22, 24 and contact points 26, 28, this forms a complete circuit. A plastic cover is then sonically welded over the recess 16 to seal the components. A layer of gel is then placed over the whole device 10; this provides an electrical contact with a user’s limb and helps keep the device adhered to a user. The gel may be protected in transit by a peelable backing layer.

The outer surface of the recess 16 is formed with integral diaphragm buttons 30 and an aperture 32 for displaying an LED. The buttons 30 are arranged to contact a corresponding button on the battery housing or PCB to activate the device. The aperture 32 displays an LED which indicates whether the device is operating.

In use the device is placed on the rear of a patient’s knee, with the positive electrode contacting the popliteal fossa. The gel provides an electrical and physical connection to the skin of the patient. When the device is activated, the control circuitry operates the electrodes according to a predefined stimulation pattern (for example, to activate the electrodes each minute using a 40 Hz pulsed DC of 20 mA for 0.1 second). These parameters are sufficient to cause the lower peroneal nerve to be stimulated. This nerve innervates both anterior and posterior muscle groups in the leg, such that they simultaneously contract. This results in isometric muscle contraction in the leg - that is, the muscle groups contract but there is little or no overall gross limb movement. The muscle contraction drives the calf muscle pump, thereby increasing blood circulation.

The present inventors have surprisingly determined that use of the device to stimulate muscle contraction in the leg can lead to improved cerebral blood circulation. The inventors believe that this alone may result in improved patient recovery from cerebral ischaemia; but further that electrical stimulation of the lower peroneal nerve may result in stimulation travelling to the spinal cord and resulting in altered electrical activity in the brain. This combination of altered electrical activity and improved cerebral blood circulation may, the inventors believe, result in improved patient outcomes post cerebral ischemia.

### Example 1 - Effect of Neuromuscular Stimulation on Brain Haemodynamics in Healthy Volunteers

A test was carried out with healthy volunteers. using a GEKO device (from FirstKind Ltd, UK). The GEKO device is a wearable electrical stimulation device designed to stimulate the lower peroneal nerve resulting in isometric muscle contractions in the leg. The GEKO device generated square wave pulses at a rate of 1 Hz. The device had 11 stimulation settings, which produced a constant current of between 27 mA and 54 mA, and pulse widths of between 35 us and 560 us (both depending on setting). Setting 1 provided the lowest stimulation; setting 11 the highest.

10 young healthy volunteers were recruited. Cerebral blood volume was measured by 16-probe fNIRS (NIRSport2). An initial resting state recording was taken for 5 minutes in sitting position, after which the GEKO device was applied on the right common peroneal nerve. The subjects were blinded from the level of stimulation. Recordings with stimulation were taken for five minutes in the sitting position, with 5 minutes rest in between each level.

FIG. 3 shows results from the monitoring in resting state, and at stimulation levels 4-11. At lower levels (4 & 5), the GEKO device increases cerebral blood volume and possibly cerebral blood flow bilaterally in the healthy subjects without any sensory stimulation. This may be explained as arterial dilatation by stimulating sensory circuit at thalamus. However, at higher level (9,10 & 11), GEKO device stimulate neurones on the contralateral hemisphere and supresses blood volume (and blood flow) to the ipsilateral hemisphere. The dose response for this effect is somewhat variable between the individuals, especially at the intermediate stimulation intensities (6, 7, 8).

The working hypothesis is that stimulation from the GEKO device can improve the outcome from stroke. The GEKO device stimulates the common peroneal nerve, which is a mixed nerve with both motor and sensory component. The motor part contracts the muscle, but it also sends signals to the brain via the sensory route. This makes the final outcome of treatment complicated as we may be sending more blood to the brain by the motor component, but if the brain cells are stimulated at the same time, the cells will consume more glucose, so the positive effect from the increased blood flow will be counterbalanced by the brain cell stimulation. Hence, we need to identify the right balance for treatment.

The experiment with healthy volunteers was carried out to prove the concept that sensory and motor stimulation works as we have hypothesised. The results confirmed that both the effect from the motor and sensory stimulation is occurring in the brain. The increased blood flow (measured by increased volume in the oxy-haemoglobin) is seen on both sides at the lower level of the stimulation (4 & 5), but at the higher level (>9) the nerve stimulation is over-powering the increased blood flow. In addition, the diffuse increase in the blood flow at the lower level suggests that the sensory pathway (by stimulating the sensory circuit at the thalamus) may be playing some role behind this findings. Otherwise, we would have seen an exponential rise in the blood flow with every level.

Therefore, we hypothesise that a lower level of stimulation (that is, lower current settings) is preferred at the acute stage of treatment, when increased blood flow is desired; while in post-acute phase, when increased neuronal activity is desired, a higher level of stimulation (higher current settings) is preferred.

### Example 2 - Future Studies

Further investigations of whether the GEKO device could increase cerebral blood flow (CBF) in patients will be undertaken. There are several different techniques available for accurately measuring cerebral blood flow. A comprehensive review on cerebral autoregulation and techniques for cerebral blood flow measurement is given in Fantini et al 2016 (Cerebral blood flow and autoregulation: current measurement techniques and prospects for noninvasive optical methods; Neurophoton. 3(3), 031411 (2016), doi: 10.1117/1.NPh.3.3.031411). Table 1 of Fantini et al describes the different techniques currently available.

We propose to use a Diffuse Optical Neuro-Monitor provided by HemoPhotonics SL, Spain. Alternative techniques may be used if suitable.

Patients will be recruited for trial on the basis of having anterior circulation stroke (ischaemic) with NIHSS of >6 (at least moderate disability), and consent sought.

CBF and CPP of patients will be assessed within the first 6 hours from the onset of stroke and then again after 24 and 48 hours, and potentially up to 72 hours. Extent of brain oedema and brain haemorrhage may also be determined.

Each test will compare several CBF measurements. CBF will be measured:
at rest (at zero degree head position) for five minutes
for five minutes immediately after applying the GEKO device
for another five minutes, 30 minutes after applying the GEKO device
for five minutes after taking off the GEKO device.

We will then compare the results to determine the effect of stimulation on CBF increase.

This protocol is intended to determine outcome of treatment during the acute phase, immediately post-stroke. Further studies may be carried out to determine outcome of treatment in the post-acute phase, for example, by monitoring glucose consumption in the brain as a measurement of neuronal activity.

1. A method of improving recovery of a patient from cerebral ischemia, the method comprising administering an electrical stimulus to a nerve of the patient innervating opposed leg muscles sufficient to cause isometric contraction of said muscles.

2. The method of claim 1, wherein said isometric contraction results in improved or increased cerebral blood flow; and/or an improvement in cerebral autoregulation; and/or an improvement in cerebral perfusion pressure (CPP).

3. The method of claim 1, wherein said electrical stimulation is sufficient to alter electrical activity in the brain.

4. A method of improving recovery of a patient from cerebral ischemia, the method comprising administering an electrical stimulus to a nerve of the patient innervating opposed leg muscles sufficient to cause isometric contraction of said muscles, said isometric contraction resulting in improved or increased cerebral blood flow, and said electrical stimulation being sufficient to alter electrical activity in the brain.

5. A method of improving recovery of a patient from cerebral ischemia, the method comprising a) administering an electrical stimulus to a nerve of the patient innervating opposed leg muscles sufficient to cause isometric contraction of said muscles, said isometric contraction resulting in improved or increased cerebral blood flow; and b) administering an electrical stimulus to a nerve of the patient innervating opposed leg muscles sufficient to cause isometric contraction of said muscles, said electrical stimulation being sufficient to alter electrical activity in the brain.

6. The method of claim 5 wherein steps a) and b) are carried out using different electrical stimulation parameters.

7. The method of claim 5 wherein steps a) and b) are carried out sequentially.

8. The method claim 1, wherein said improvement in recovery is a reduction in cerebral cell death; and/or an improvement in cerebral autoregulation; and/or an improvement in cerebral perfusion pressure (CPP).

9. The method of claim 3, wherein said improvement in recovery is an increase in neuroplasticity; and/or an improvement in cognitive function.

10. The method of claim 2, wherein the improved or increased cerebral blood flow is an improvement in cerebral autoregulation; and/or an improvement in cerebral perfusion pressure (CPP).

11. A method for selecting a patient for therapy to aid recovery from cerebral ischaemia, the method comprising:
identifying a patient who has experienced cerebral ischaemia;
administering an electrical stimulus to a nerve of the patient innervating opposed leg muscles sufficient to cause isometric contraction of said muscles;
monitoring cerebral blood flow prior to and during and/or after said administration of electrical stimulus; and
selecting the patient for continued therapy in the event that cerebral blood flow increases during and/or after said administration.

12. A method for selecting a patient for therapy to aid recovery from cerebral ischaemia, the method comprising:
identifying a patient who has experienced cerebral ischaemia;
administering an electrical stimulus to a nerve of the patient innervating opposed leg muscles sufficient to cause isometric contraction of said muscles;
monitoring brain electrical activity prior to and during and/or after said administration of electrical stimulus; and
selecting the patient for continued therapy in the event that brain electrical activity alters during and/or after said administration.

13. A method for selecting a patient for therapy to aid recovery from cerebral ischaemia, the method comprising:
identifying a patient who has experienced cerebral ischaemia;
administering an electrical stimulus to a nerve of the patient innervating opposed leg muscles sufficient to cause isometric contraction of said muscles;
monitoring cerebral blood flow and brain electrical activity prior to and during and/or after said administration of electrical stimulus; and
selecting the patient for continued therapy in the event that cerebral blood flow increases and brain electrical activity alters during and/or after said administration.
